(19) United States
(12) Patent Application Publication
(10) Pub. No.: US 2017/0281372 A1
(43) Pub. Date: Oct. 5, 2017

(54) APPARATUS AND METHOD FOR A SPLIT TOE BLADE

(71) Applicant: Fillauer Composites LLC, Salt Lake City, UT (US)

(72) Inventors: Eric Rubie, Salt Lake City, UT (US); David Rotter, Chicago, IL (US); Jack Stites, Salt Lake City, UT (US); Jon Winegar, Farmington, UT (US)

(73) Assignee: Fillauer Composites LLC, Salt Lake City, UT (US)

(21) Appl. No.: 15/477,856

(22) Filed: Apr. 3, 2017

Related U.S. Application Data
(60) Provisional application No. 62/317,909, filed on Apr. 4, 2016.

(51) Int. Cl. A61F 2/66

(52) U.S. Cl. CPC A61F 2/66; A61F 2002/6614; A61F 2002/6621; A61F 2002/6671; A61F 2002/6678

(57) ABSTRACT

A split toe blade comprising a medial toe member, a lateral toe member that is disposed adjacent to the medial toe member, and a connecting web that is disposed between the medial toe member and the lateral toe member. The preferred connecting web controls the amount of traverse compliance in the event an off-axis load is applied to the blade. A method comprising providing a split toe blade and applying an off-axis force to the blade.

### CROSS-REFERENCES TO RELATED APPLICATIONS/PATENTS

This application relates back to and claims the benefit of priority from U.S. Provisional Application for Patent Ser. No. 62/317,909 titled “Split Toe Blade” and filed on Apr. 4, 2016.

### FIELD OF THE INVENTION

The present invention relates generally to apparatuses and methods for a lower limb prosthetic device, and particularly to apparatuses and methods for split toe blades.

### BACKGROUND AND DESCRIPTION OF THE PRIOR ART

It is known to use apparatuses and methods for lower limb prosthetic devices, including running blades. Conventional apparatuses and methods, however, suffer from one or more disadvantages. For example, conventional running blade apparatuses and methods do not provide sufficient multi-axial performance and are undesirably rigid in the transverse plane. As a result, conventional solid toe running blade apparatuses and methods are not suited for use on uneven terrains or other non-running activities. In addition, conventional solid toe running blades do not allow the user to turn around easily or run around corners quickly. Conventional split toe feet apparatuses and methods are not suited for running activities because they are undesirably heavy.

It would be desirable, therefore, if an apparatus and method for a split toe blade could be provided that would provide sufficient multi-axial performance and sufficient flexibility in the transverse plane. It would also be desirable if such an apparatus and method for a split toe blade could be provided that would be suitable for use on uneven terrain and other non-running activities. In addition, it would be desirable is such an apparatus and method for a split toe blade could be provided that would allow the user to turn around easily and run faster around corners. It would be further desirable if such an apparatus and method for a split toe blade could be provided that would control the amount of traverse compliance and prevent the different toe portions of the blade from overlapping in the event an off-axis load is applied.

### ADVANTAGES OF THE PREFERRED EMBODIMENTS OF THE INVENTION

Accordingly, it is an advantage of the preferred embodiments of the invention claimed herein to provide an apparatus and method for a split toe blade that provides sufficient multi-axial performance and sufficient flexibility in the transverse plane. It is also an advantage of the preferred embodiments of the invention claimed herein to provide an apparatus and method for a split toe blade that is well-suited for running on uneven terrain and other non-running activities. In addition, it is an advantage of the preferred embodiments of the invention claimed herein to provide an apparatus and method for a split toe blade that allows the user to turn around easily and run faster around corners. It is another advantage of the preferred embodiments of the invention claimed herein to provide an apparatus and method for a split toe blade that controls the amount of traverse compliance and prevents the different toe portions of the blade from overlapping in the event an off-axis load is applied.

Additional advantages of the preferred embodiments of the invention will become apparent from an examination of the drawings and the ensuing description.

### SUMMARY OF THE INVENTION

The apparatus of the invention comprises a split toe blade. The preferred split toe blade comprises a medial toe member, a lateral toe member that is disposed adjacent to the medial toe member, and a connecting web that is disposed between the medial toe member and the lateral toe member. In the preferred embodiments of the split toe blade, the connecting web controls the amount of traverse compliance in the event an off-axis load is applied to the blade.

The method of the invention comprises a method for controlling the movement of a split toe blade. The preferred method comprises the step of providing a split toe blade. The preferred split toe blade comprises a medial toe member, a lateral toe member that is disposed adjacent to the medial toe member, and a connecting web that is disposed between the medial toe member and the lateral toe member. In the preferred embodiments of the split toe blade, the connecting web controls the amount of traverse compliance in the event an off-axis load is applied to the blade. The preferred method also comprises the step of applying an off-axis force to the blade.

### BRIEF DESCRIPTION OF THE DRAWINGS

The presently preferred embodiments of the invention are illustrated in the accompanying drawings, in which like reference numerals represent like parts throughout, and in which:

FIG. 1 is a front perspective view of the preferred embodiment of the split toe blade in accordance with the present invention.

FIG. 2 is a back perspective view of the preferred split toe blade illustrated in FIG. 1.

FIG. 3 is a right side view of the preferred split toe blade illustrated in FIGS. 1-2.

FIG. 4 is a right side view of the preferred split toe blade illustrated in FIGS. 1-3 with a vertical load applied to it.

FIG. 5 is a front view of the preferred split toe blade illustrated in FIGS. 1-4 with an off-axis load applied to it.

FIG. 6 is a bottom view of the preferred split toe blade illustrated in FIGS. 1-5.

FIG. 7 is a front perspective view of the preferred toe sole illustrated in FIGS. 1-6.

FIG. 8 is a top view of the preferred toe sole illustrated in FIGS. 1-7.

FIG. 9 is a front view of the preferred toe sole illustrated in FIGS. 1-8

FIG. 10 is a front perspective view of a first alternative embodiment of the split toe blade in accordance with the present invention.

FIG. 11 is a front view of the first alternative embodiment of the split toe blade illustrated in FIG. 10.

FIG. 12 is a back view of the first alternative embodiment of the split toe blade illustrated in FIGS. 10-11.

FIG. 13 is a right side view of the first alternative embodiment of the split toe blade illustrated in FIGS. 10-12.

FIG. 14 is a front perspective view of a second alternative embodiment of the split toe blade in accordance with the present invention.

FIG. 15 is a right side view of the second alternative embodiment of the split toe blade illustrated in FIG. 14.

FIG. 16 is a front view of the second alternative embodiment of the split toe blade illustrated in FIGS. 14-15.

FIG. 17 is a back view of the second alternative embodiment of the split toe blade illustrated in FIGS. 14-16.

FIG. 18 is a front perspective view of a third alternative embodiment of the split toe blade in accordance with the present invention.

FIG. 19 is right side view of the third alternative embodiment of the split toe blade illustrated in FIG. 18.

FIG. 20 is a front view of the third alternative embodiment of the split toe blade illustrated in FIGS. 18-19.

FIG. 21 is a back view of the third alternative embodiment of the split toe blade illustrated in FIGS. 18-20.

FIG. 22 is a front perspective view of a fourth alternative embodiment of the split toe blade in accordance with the present invention.

FIG. 23 is a right side view of the fourth alternative embodiment of the split toe blade illustrated in FIG. 22.

FIG. 24 is a back view of the fourth alternative embodiment of the split toe blade illustrated in FIGS. 22-23.

FIG. 25 is a bottom view of the fourth alternative embodiment of the split toe blade illustrated in FIGS. 22-24.

### DESCRIPTION OF THE PREFERRED EMBODIMENTS OF THE INVENTION

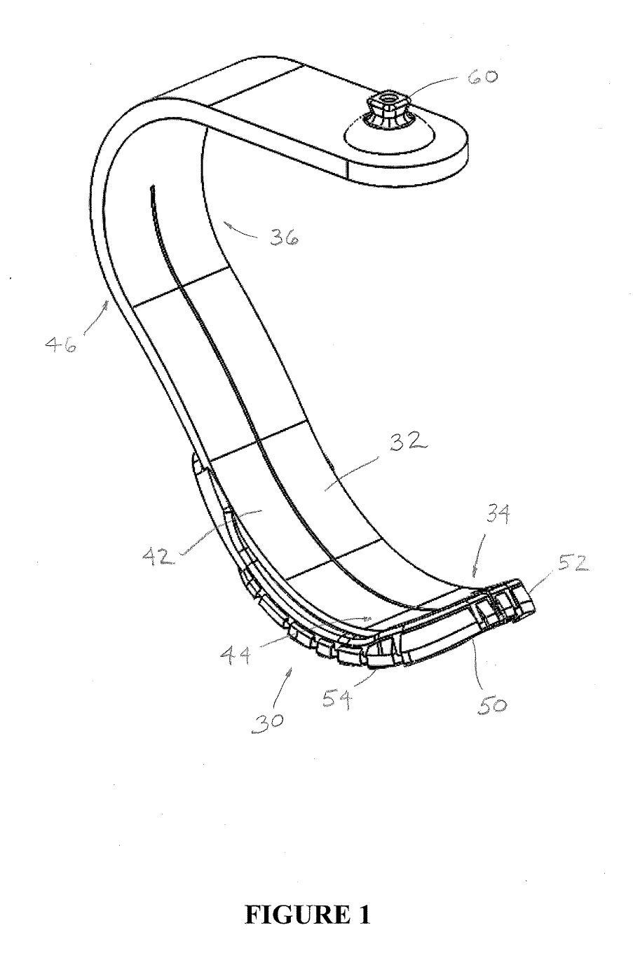

Referring now to the drawings, the preferred embodiments of the apparatus and method for a split toe blade in accordance with the present invention are illustrated by FIGS. 1 through 21. As shown in FIGS. 1-21, the preferred embodiments of the apparatus and method for a split to blade are adapted to provide sufficient multi-axial performance and sufficient flexibility in the transverse plane. The preferred embodiments of the apparatus and method for a split toe blade are also adapted to provide a split toe blade that is well-suited for running on uneven terrain and other non-running activities. In addition, the preferred embodiments of the apparatus and method for a split toe blade are adapted to allow the user to turn around easily and run faster around corners. The preferred embodiments of the apparatus and method for a split toe blade are further adapted to prevent the different toe portions of the blade from overlapping in the event an off-axis load is applied.

Referring now to FIG. 1, a front perspective view of the preferred embodiment of the split toe blade in accordance with the present invention is illustrated. As shown in FIG. 1, the preferred split toe blade is designated generally by reference numeral 30. Preferred split toe blade 30 comprises medial toe member 32 having forward end 34 and rearward end 36. Preferred split toe blade 30 also comprises lateral toe member 42 having forward end 44 and rearward end 46. Preferred lateral toe member 42 is disposed adjacent to preferred medial toe member 32.

Still referring to FIG. 1, preferred split toe blade 30 also comprises toe sole 50. Preferred toe sole 50 is disposed on at least a portion of medial toe member 32 and on at least a portion of lateral toe member 42. Preferably, toe sole 50 is disposed around at least a portion of medial toe member 32 and around at least a portion of lateral toe member 42. Preferred toe sole 50 comprises an elastomeric material and includes medial toe sole member 52 and lateral toe sole member 54.

Still referring to FIG. 1, preferred split toe blade 30 further comprises a mounting means. The preferred mounting means comprises standard modular mount 60. It is contemplated within the scope of the invention, however, that the mounting means may comprise any suitable type of mounting means such as a posterior mount and the like.

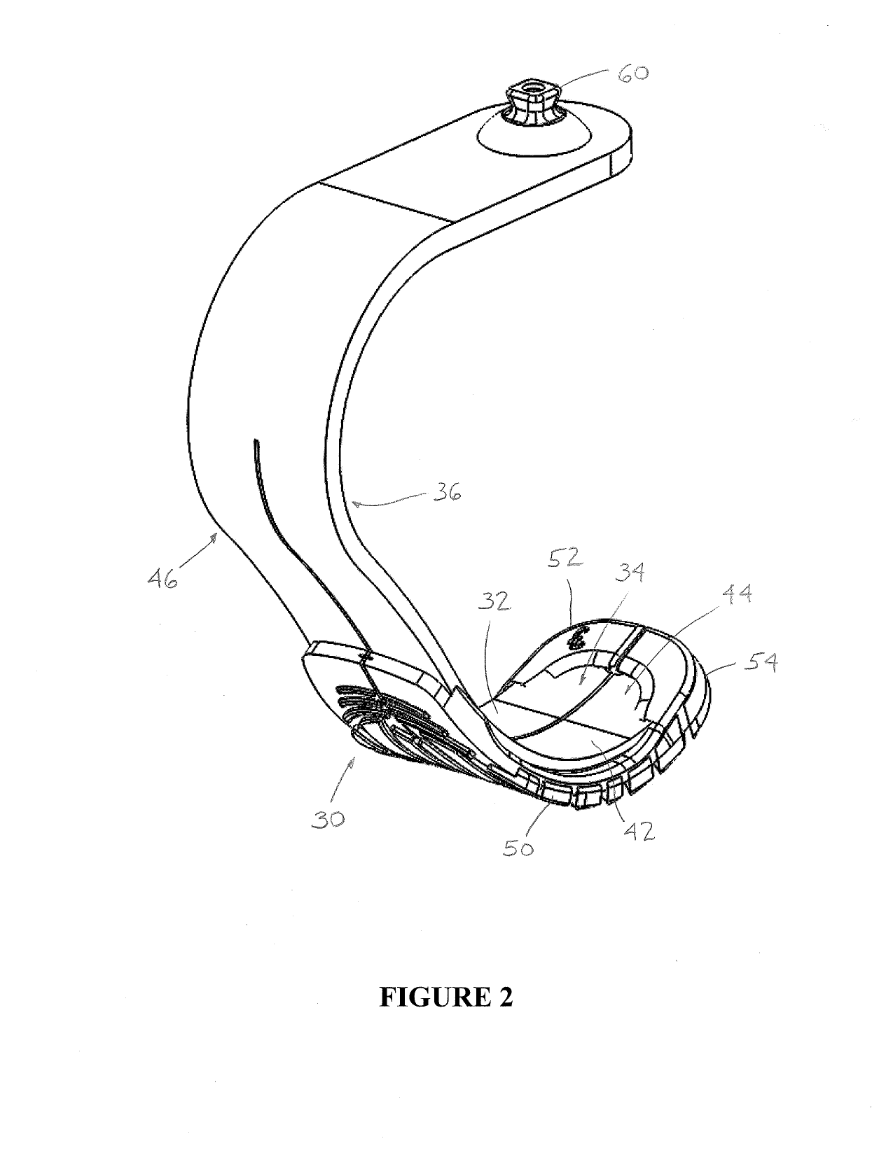

Referring now to FIG. 2, a back perspective view of preferred split toe blade 30 is illustrated. As shown in FIG. 2, preferred split toe blade 30 comprises medial toe member 32 having forward end 34 and rearward end 36, lateral toe member 42 having forward end 44 and rearward end 46, toe sole 50 having medial toe sole member 52 and lateral toe sole member 54, and standard modular mount 60.

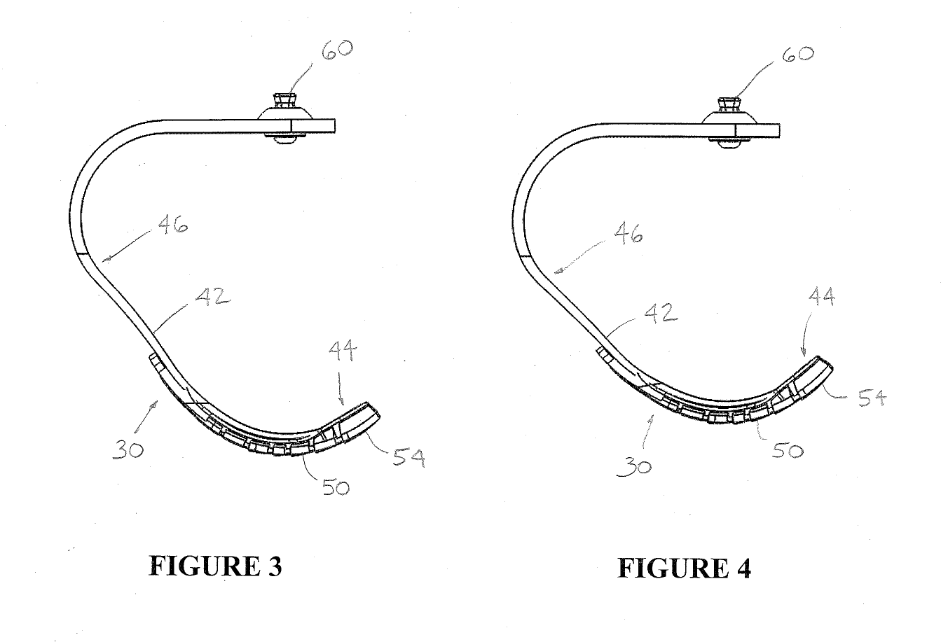

Referring now to FIG. 3, a right side view of preferred split toe blade 30 is illustrated. As shown in FIG. 3, preferred split toe blade 30 comprises lateral toe member 42 having forward end 44 and rearward end 46, toe sole 50 having lateral toe sole member 54, and standard modular mount 60.

Referring now to FIG. 4, a right side view of preferred split toe blade 30 is illustrated with a vertical load applied to it. As shown in FIG. 4, preferred split toe blade 30 comprises lateral toe member 42 having forward end 44 and rearward end 46, toe sole 50 having lateral toe sole member 54, and standard modular mount 60. As FIG. 4 illustrates, preferred split toe blade 30 compresses when a vertical load is applied to it.

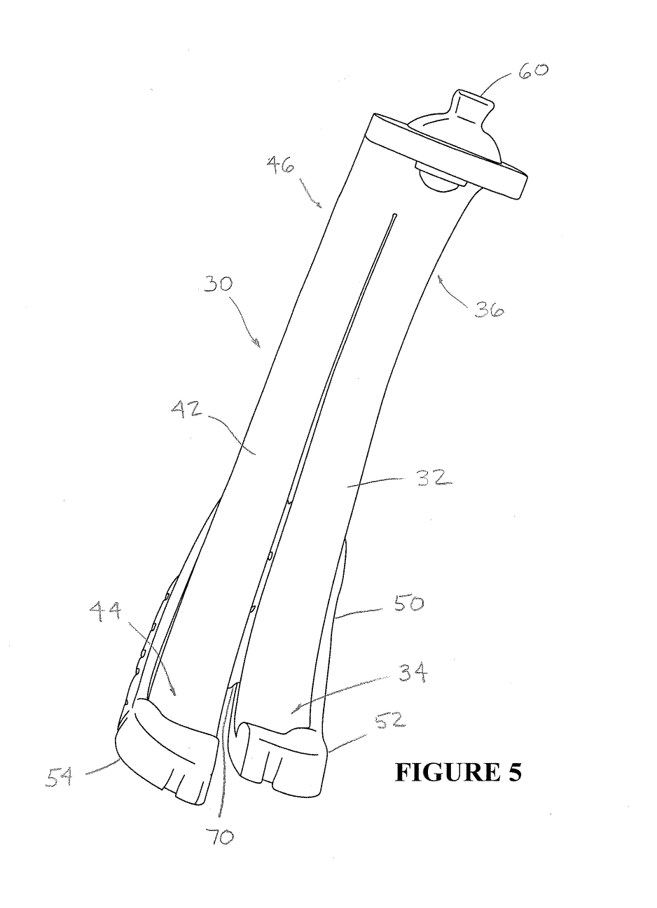

Referring now to FIG. 5, a front view of preferred split toe blade 30 is illustrated with an off-axis load applied to it. As shown in FIG. 5, preferred split toe blade 30 comprises medial toe member 32 having forward end 34 and rearward end 36, lateral toe member 42 having forward end 44 and rearward end 46, toe sole 50 having medial toe sole member 52 and lateral toe sole member 54, and standard modular mount 60. As further shown in FIG. 5, preferred split toe blade 30 also comprises connecting web 70. Preferred connecting web 70 is disposed between preferred medial toe member 32 and preferred lateral toe member 42. Preferably, connecting web 70 controls the amount of traverse compliance and prevents medial toe member 32 and lateral toe member 42 from overlapping in the event an off-axis load is applied to blade 30. More particularly, preferred connecting web 70 is adapted to control the amount of traverse compliance, i.e. medial-lateral flexibility, so as to influence the user's ability to walk or run on uneven terrain. Traverse compliance, in this situation, encompasses frontal plane motion in both inversion and eversion where the foot is flat on a surface and a pronation/supination set-up where more weight is to the front of the foot. Preferred connecting web 70 comprises an elastomeric material, however, it is contemplated within the scope of the invention that the connecting web may comprise any suitable material or combination of materials.

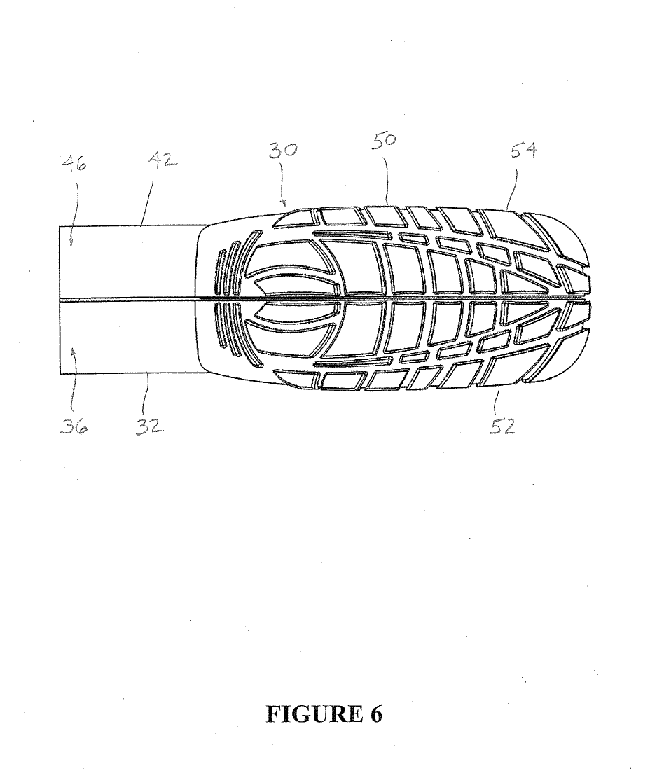

Referring now to FIG. 6, a bottom view of preferred split toe blade 30 is illustrated. As shown in FIG. 6, preferred split toe blade 30 comprises medial toe member 32 having rearward end 36, lateral toe member 42 having rearward end 46, and toe sole 50 having medial toe sole member 52 and lateral toe sole member 54. While FIGS. 1-6 illustrate the preferred configuration and arrangement of split toe blade 30, it is contemplated within the scope of the invention that the split toe blade may be of any suitable configuration and arrangement.

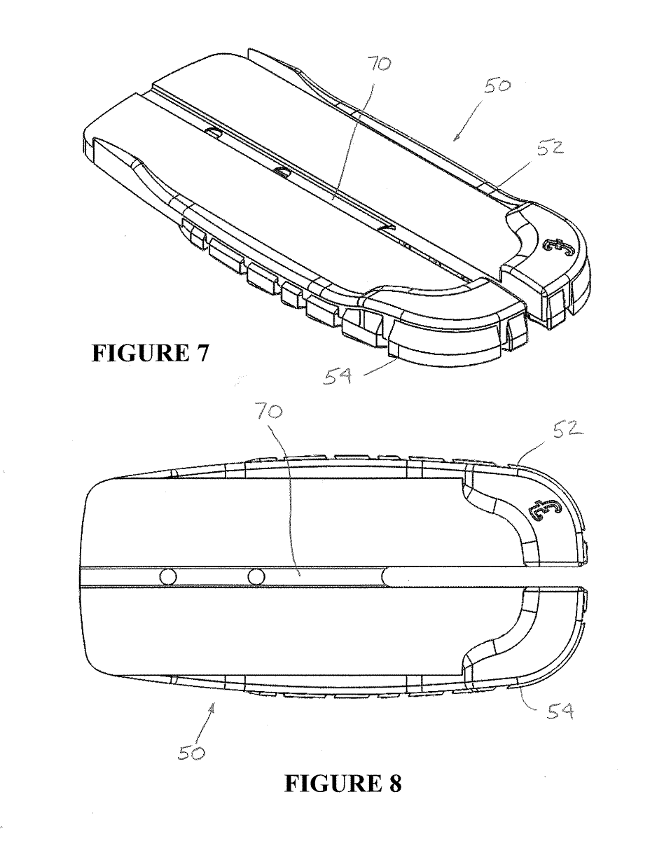

Referring now to FIG. 7, a front perspective view of preferred toe sole 50 is illustrated. As shown in FIG. 7, preferred toe sole 50 comprises medial toe sole member 52, lateral toe sole member 54, and connecting web 70.

Referring now to FIG. 8, a top view of preferred toe sole 50 is illustrated. As shown in FIG. 8, preferred toe sole 50 comprises medial toe sole member 52, lateral toe sole member 54, and connecting web 70.

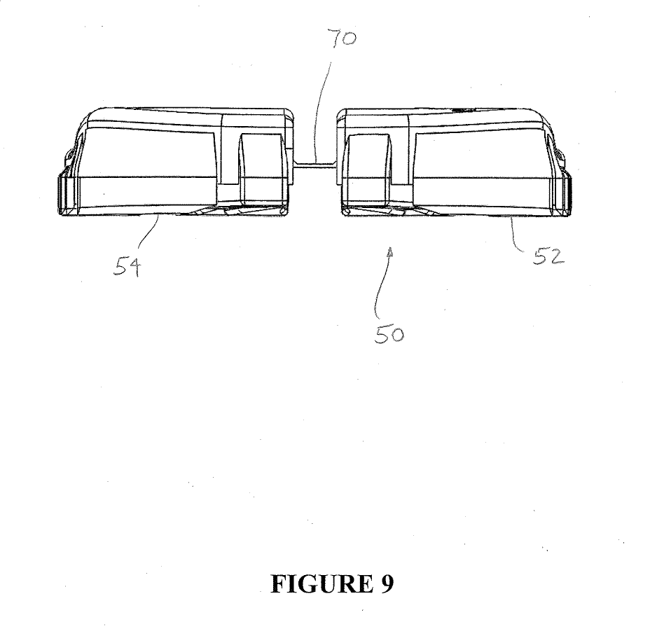

Referring now to FIG. 9, a front view of preferred toe sole 50 is illustrated. As shown in FIG. 9, preferred toe sole 50 comprises medial toe sole member 52, lateral toe sole member 54, and connecting web 70. Whiles FIGS. 7-9 illustrate the preferred configuration and arrangement of sole toe 50, it is contemplated within the scope of the invention that the sole toe may be of any suitable configuration and arrangement. More particularly, it is contemplated within the scope of the invention that the connecting web may be a distinct component, separate from the toe sole, and the size and disposition of the connecting web may be altered to accommodate different loads.

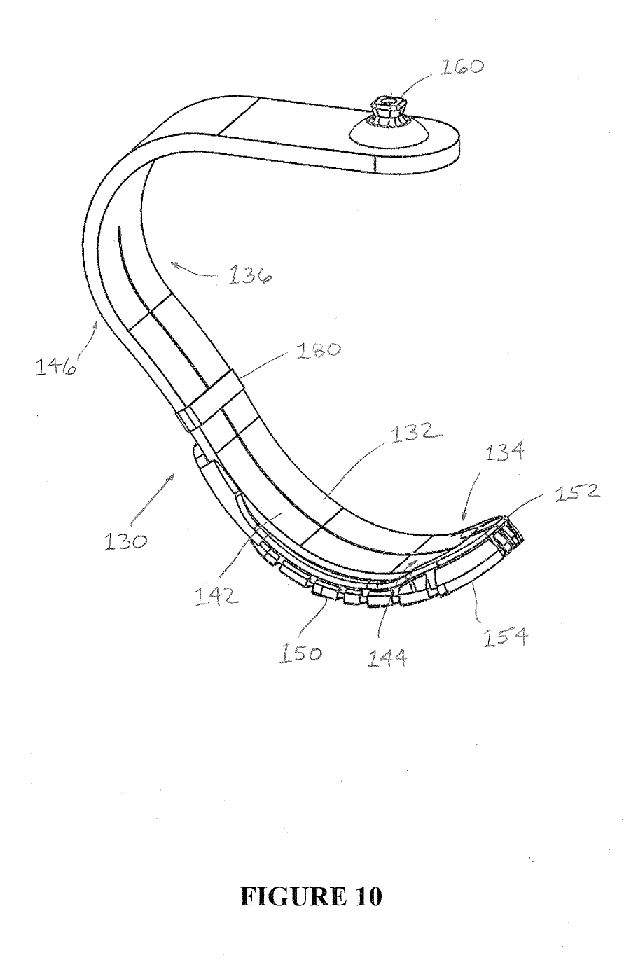

Referring now to FIG. 10, a front perspective view of a first alternative embodiment of the split toe blade in accordance with the present invention is illustrated. As shown in FIG. 10, the preferred split toe blade is designated generally by reference numeral 130. Preferred split toe blade 130 comprises medial toe member 132 having forward end 134 and rearward end 136. Preferred split toe blade 130 also comprises lateral toe member 142 having forward end 144 and rearward end 146. Preferred lateral toe member 142 is disposed adjacent to preferred medial toe member 132.

Still referring to FIG. 10, preferred split toe blade 130 also comprises toe sole 150. Preferred toe sole 150 is disposed on at least a portion of medial toe member 132 and on at least a portion of lateral toe member 142. Preferably, toe sole 150 is disposed around at least a portion of medial toe member 132 and around at least a portion of lateral toe member 142. Preferred toe sole 150 comprises an elastomeric material and includes medial toe sole member 152 and lateral toe sole member 154.

Still referring to FIG. 10, preferred split toe blade 130 further comprises a mounting means. The preferred mounting means comprises standard modular mount 160. It is contemplated within the scope of the invention, however, that the mounting means may comprise any suitable type of mounting means such as a posterior mount and the like.

Still referring to FIG. 10, preferred split toe blade 130 still further comprises band 180. Preferred band 180 comprises an elastomeric material and polyethylene terephthalate. It is contemplated within the scope of the invention, however, that the band may be made from any suitable material or combination of materials. It is also contemplated within the scope of the invention that the size and disposition of the band may altered to accommodate different loads and that more than one band may be used.

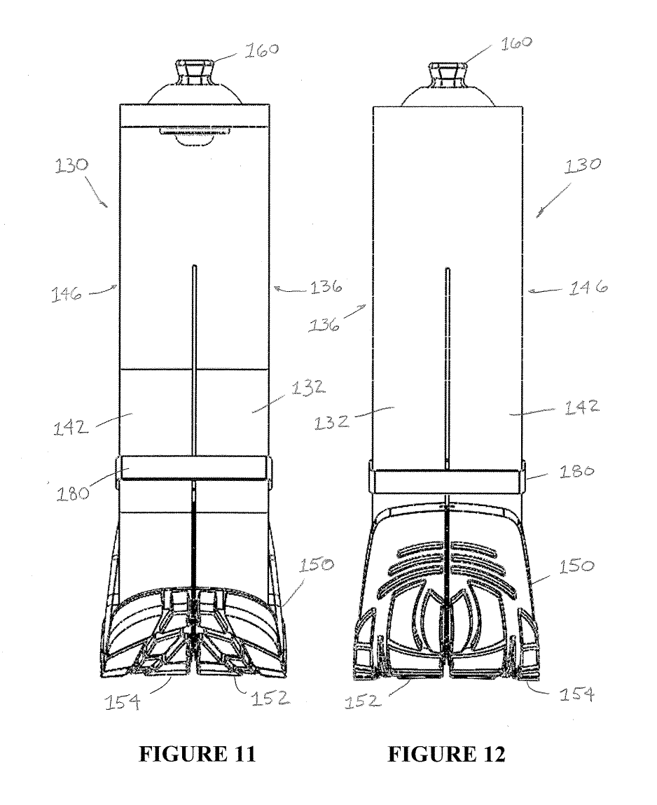

Referring now to FIG. 11, a front view of preferred split toe blade 130 is illustrated. As shown in FIG. 11, preferred split toe blade 130 comprises medial toe member 132 having rearward end 136, lateral toe member 142 having rearward end 146, toe sole 150 having medial toe sole member 152 and lateral toe sole member 154, standard modular mount 160, and band 180.

Referring now to FIG. 12, a back view of preferred split toe blade 130 is illustrated. As shown in FIG. 12, preferred split toe blade 130 comprises medial toe member 132 having rearward end 136, lateral toe member 142 having rearward end 146, toe sole 150 having medial toe sole member 152 and lateral toe sole member 154, standard modular mount 160, and band 180.

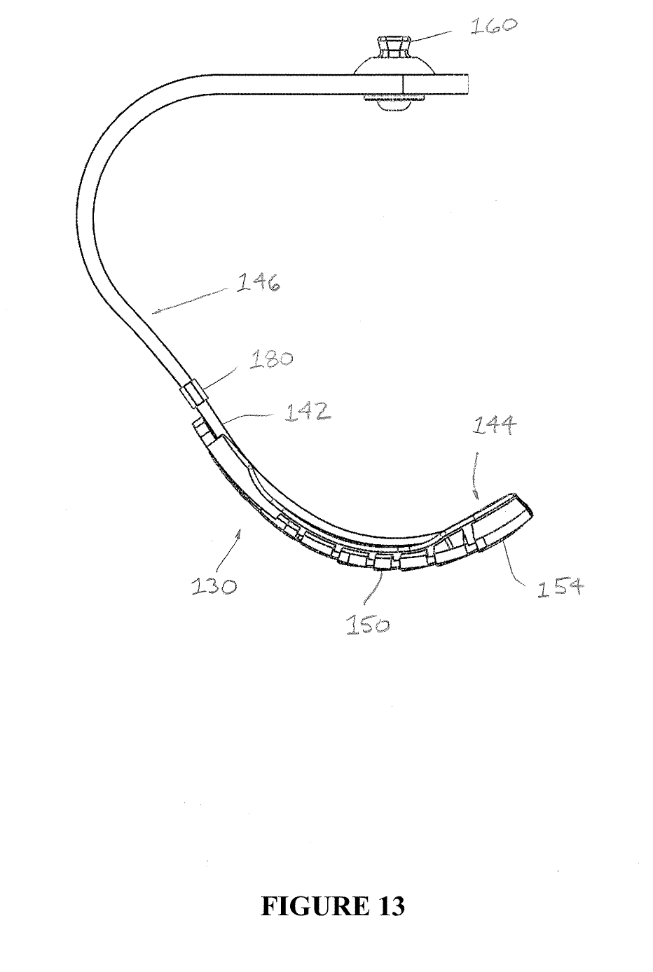

Referring now to FIG. 13, a right side view of preferred split toe blade 130 is illustrated. As shown in FIG. 13, preferred split toe blade 130 comprises lateral toe member 142 having forward end 144 and rearward end 146, toe sole 150 having lateral toe sole member 154, standard modular mount 160, and band 180. While FIGS. 10-13 illustrate the preferred configuration and arrangement of split toe blade 130, it is contemplated within the scope of the invention that the split toe blade may be of any suitable configuration and arrangement.

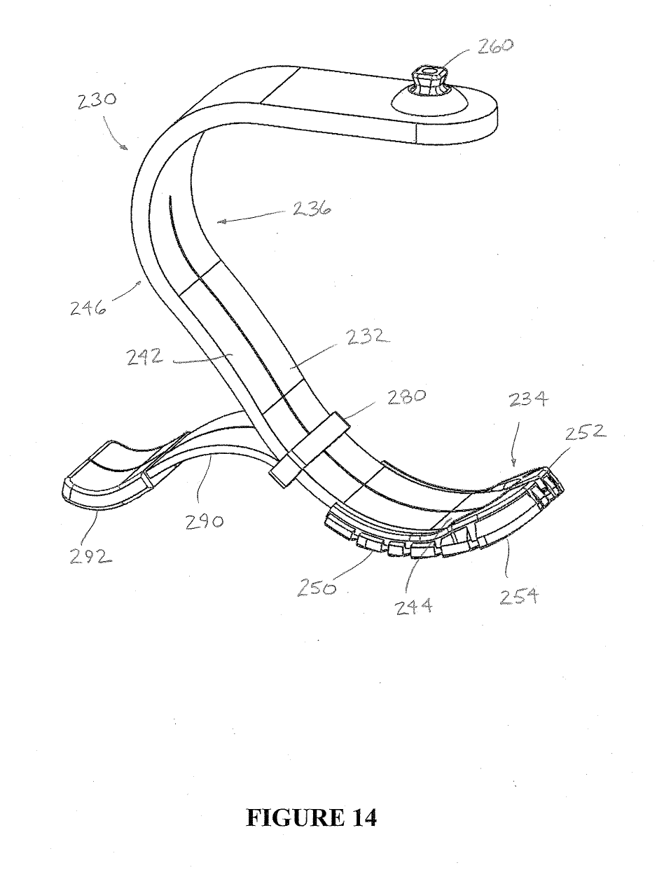

Referring now to FIG. 14, a front perspective view of a second alternative embodiment of the split toe blade in accordance with the present invention is illustrated. As shown in FIG. 14, the preferred split toe blade is designated generally by reference numeral 230. Preferred split toe blade 230 comprises medial toe member 232 having forward end 234 and rearward end 236. Preferred split toe blade 230 also comprises lateral toe member 242 having forward end 244 and rearward end 246. Preferred lateral toe member 242 is disposed adjacent to preferred medial toe member 232.

Still referring to FIG. 14, preferred split toe blade 230 also comprises toe sole 250. Preferred toe sole 250 is disposed on at least a portion of medial toe member 232 and on at least a portion of lateral toe member 242. Preferably, toe sole 250 is disposed around at least a portion of medial toe member 232 and around at least a portion of lateral toe member 242. Preferred toe sole 250 comprises an elastomeric material and includes medial toe sole member 252 and lateral toe sole member 254.

Still referring to FIG. 14, preferred split toe blade 230 further comprises a mounting means. The preferred mounting means comprises standard modular mount 260. It is contemplated within the scope of the invention, however, that the mounting means may comprise any suitable type of mounting means such as a posterior mount and the like.

Still referring to FIG. 14, preferred split toe blade 230 still further comprises band 280. Preferred band 180 comprises and elastomeric material and polyethylene terephthalate. It is contemplated within the scope of the invention, however, that the band may be made from any suitable material or combination of materials.

Still referring to FIG. 14, preferred split toe blade 230 also comprises heel member 290 and heel sole 292 which is disposed on at least a portion of the heel member. Preferably, heel sole 292 is disposed around at least a portion of heel member 290.

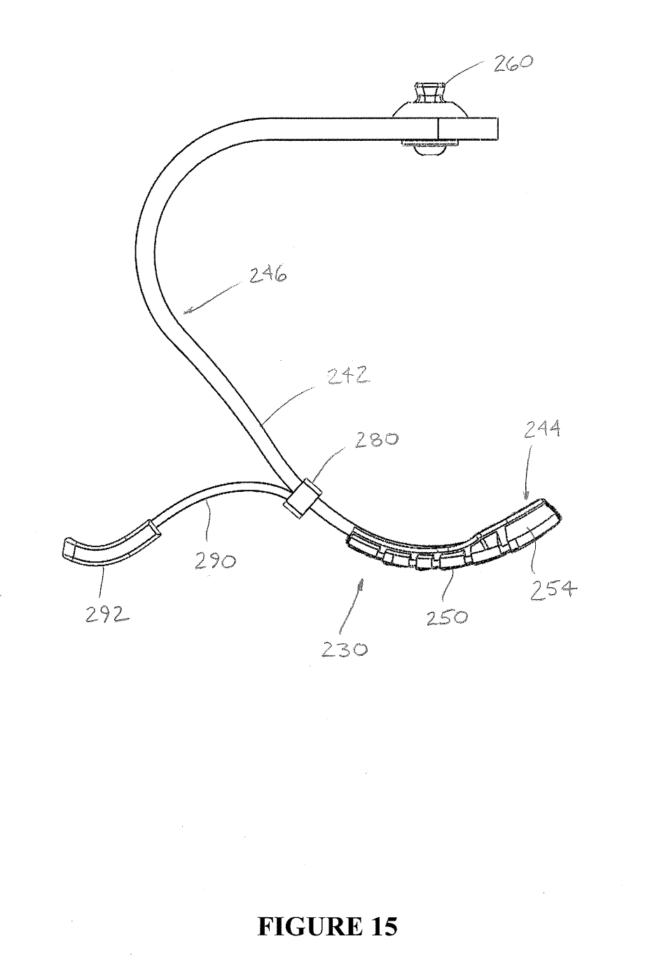

Referring now to FIG. 15, a right side view of preferred split toe blade 230 is illustrated. As shown in FIG. 15, preferred split toe blade 230 comprises lateral toe member 242 having forward end 244 and rearward end 246, toe sole 250 having lateral toe sole member 254, standard modular mount 260, band 280, heel member 290, and heel sole 292.

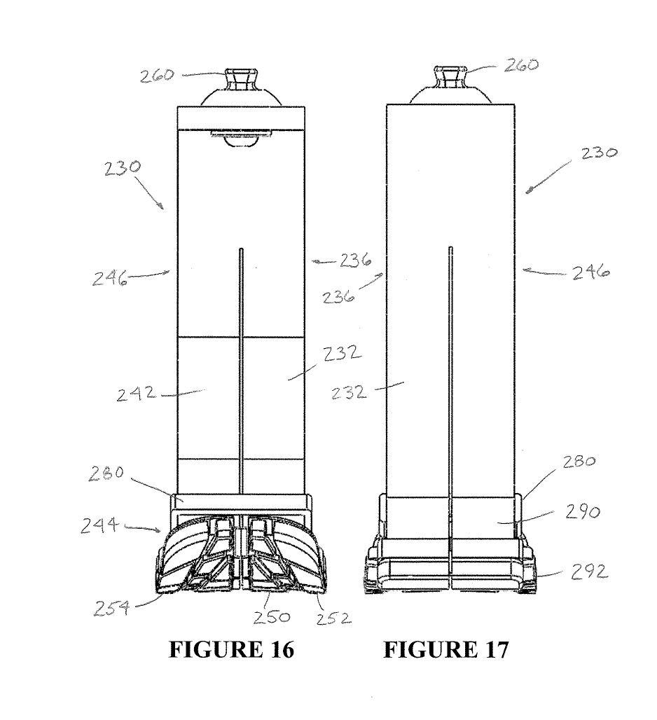

Referring now to FIG. 16, a front view of preferred split toe blade 230 is illustrated. As shown in FIG. 16, preferred split toe blade 230 comprises medial toe member 232 having rearward end 236, lateral toe member 242 having rearward end 246, toe sole 250 having medial toe sole member 252 and lateral toe sole member 254, standard modular mount 260, and band 280.

Referring now to FIG. 17, a back view of preferred split toe blade 230 is illustrated. As shown in FIG. 17, preferred split toe blade 230 comprises medial toe member 232 having rearward end 236, lateral toe member 242 having rearward end 246, standard modular mount 260, band 280, heel member 290, and heel sole 292. While FIGS. 14-17 illustrate the preferred configuration and arrangement of split toe blade 230, it is contemplated within the scope of the invention that the split toe blade may be of any suitable configuration and arrangement.

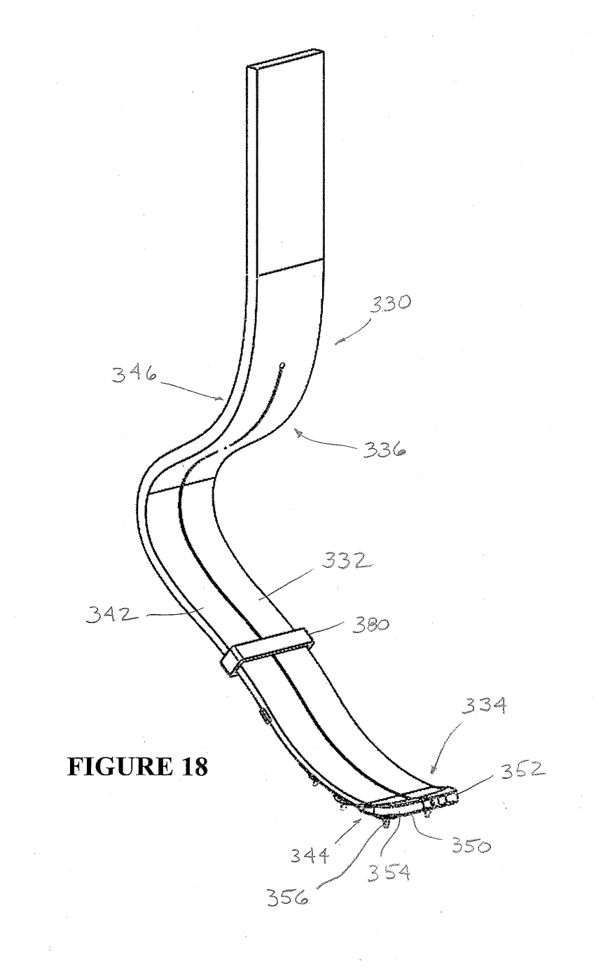

Referring now to FIG. 18, a front perspective view of a third alternative embodiment of the split toe blade in accordance with the present invention is shown. As shown in FIG. 18, the preferred split toe blade is designated generally by reference numeral 330. Preferred split toe blade 330 comprises medial toe member 332 having forward end 334 and rearward end 336. Preferred split toe blade 330 also comprises lateral toe member 342 having forward end 344 and rearward end 346. Preferred lateral toe member 342 is disposed adjacent to preferred medial toe member 332.

Still referring to FIG. 18, preferred split toe blade 330 also comprises toe sole 350. Preferred toe sole 350 is disposed on at least a portion of medial toe member 332 and on at least a portion of lateral toe member 342. Preferably, toe sole 350 is disposed around at least a portion of medial toe member 332 and around at least a portion of lateral toe member 342. Preferred toe sole 350 comprises an elastomeric material and includes medial toe sole member 352 and lateral toe sole member 354. Preferred toe sole 350 also comprises at least one toe spike 356.

Still referring to FIG. 18, preferred split toe blade 330 still further comprises band 380. Preferred band 380 comprises an elastomeric material and polyethylene terephthalate. It is contemplated within the scope of the invention, however, that the band may be made from any suitable material or combination of materials.

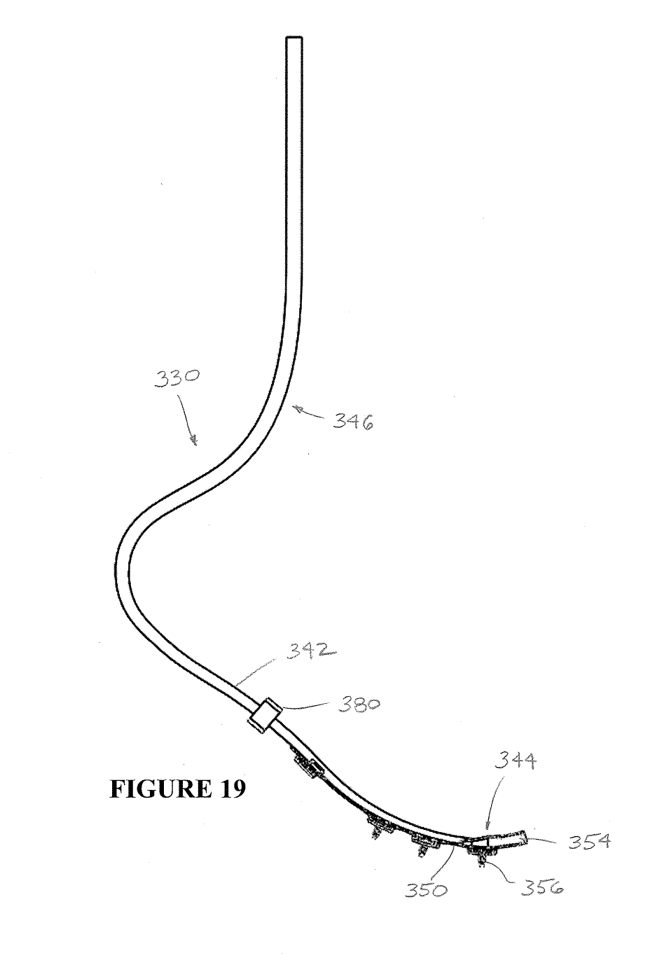

Referring now to FIG. 19, a right side view of preferred split toe blade 330 is illustrated. As shown in FIG. 19, preferred split toe blade 330 comprises lateral toe member 342 having forward end 344 and rearward end 346, toe sole 350 having lateral toe sole member 354 and toe spikes 356, and band 380.

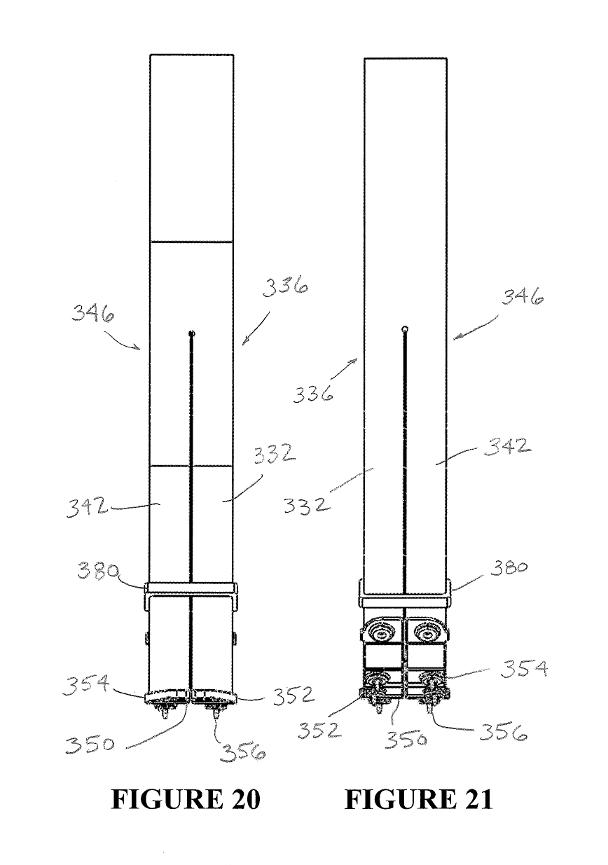

Referring now to FIG. 20, a front view of preferred split toe blade 330 is illustrated. As shown in FIG. 20, preferred split toe blade 330 comprises medial toe member 332 having rearward end 336, lateral toe member 342 having rearward end 346, toe sole 350 having medial toe sole member 352, lateral toe sole member 354 and toe spikes 356, and band 380.

Referring now to FIG. 21, a back view of preferred split toe blade 330 is illustrated. As shown in FIG. 21, preferred split toe blade 330 comprises medial toe member 332 having rearward end 336, lateral toe member 342 having rearward end 346, toe sole 350 having medial toe sole member 352 and lateral toe sole member 354, and toe spikes 356, and band 380. While FIGS. 18-21 illustrate the preferred configuration and arrangement of split toe blade 330, it is contemplated within the scope of the invention that the split toe blade may be of any suitable configuration and arrangement.

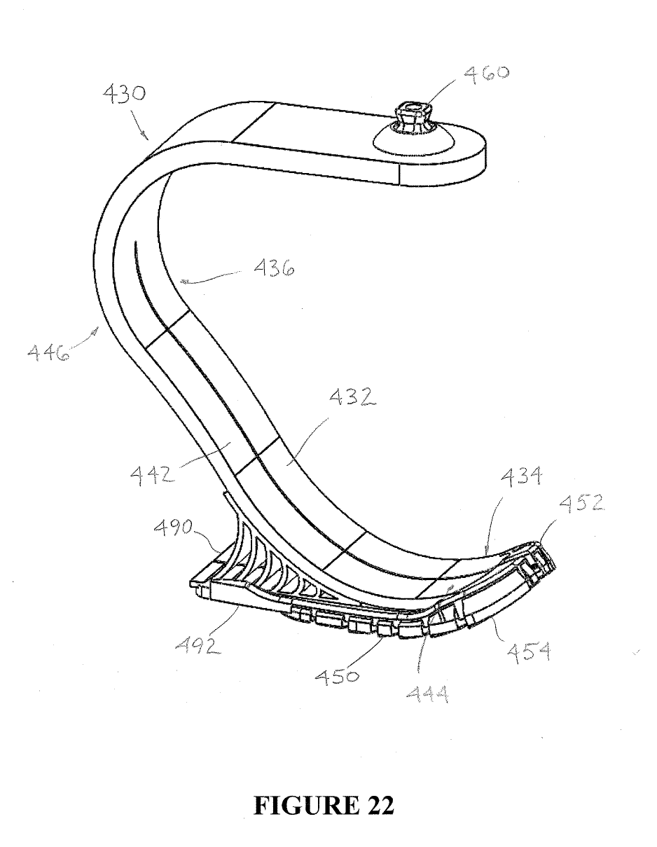

Referring now to FIG. 22, a front perspective view of a fourth alternative embodiment of the split toe blade in accordance with the present invention is illustrated. As shown in FIG. 22, the preferred split toe blade is designated generally by reference numeral 430. Preferred split toe blade 430 comprises medial toe member 432 having forward end 434 and rearward end 436. Preferred split toe blade 430 also comprises lateral toe member 442 having forward end 444 and rearward end 446. Preferred lateral toe member 442 is disposed adjacent to preferred medial toe member 432.

Still referring to FIG. 22, preferred split toe blade 430 also comprises toe sole 450. Preferred toe sole 450 is disposed on at least a portion of medial toe member 432 and on at least a portion of lateral toe member 442. Preferably, toe sole 450 is disposed around at least a portion of medial toe member 432 and around at least a portion of lateral toe member 442. Preferred toe sole 450 comprises an elastomeric material and includes medial toe sole member 452 and lateral toe sole member 454.

Still referring to FIG. 22, preferred split toe blade 430 further comprises a mounting means. The preferred mounting means comprises standard modular mount 460. It is contemplated within the scope of the invention, however, that the mounting means may comprise any suitable type of mounting means such as a posterior mount and the like.

Still referring to FIG. 22, preferred split toe blade 430 also comprises heel member 490 and heel sole 492 which is disposed on at least a portion of the heel member. Preferably, heel sole 492 is disposed around at least a portion of heel member 490.

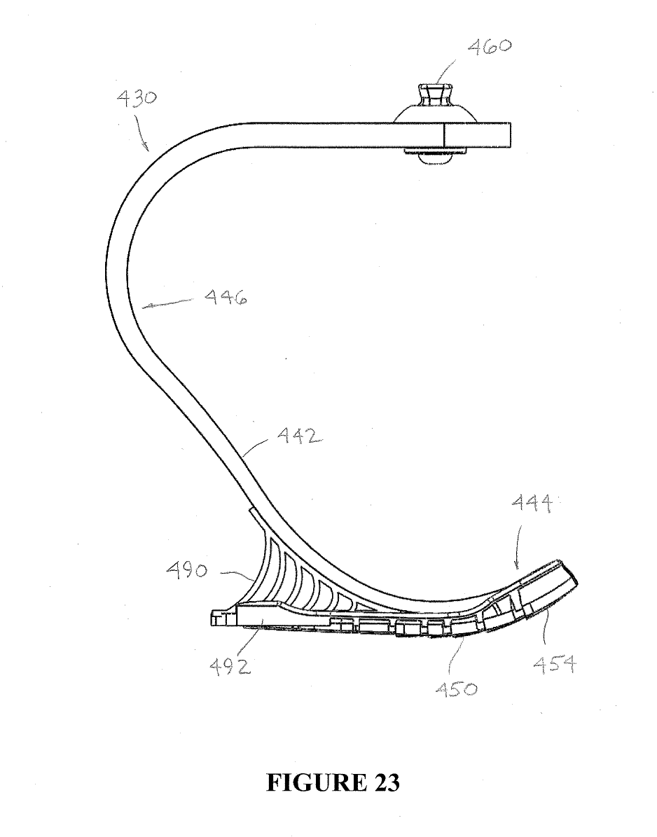

Referring now to FIG. 23, a right side view of preferred split toe blade 430 is illustrated. As shown in FIG. 23, preferred split toe blade 430 comprises lateral toe member 442 having forward end 444 and rearward end 446, toe sole 450 having lateral toe sole member 454, standard modular mount 460, heel member 490 and heel sole 492.

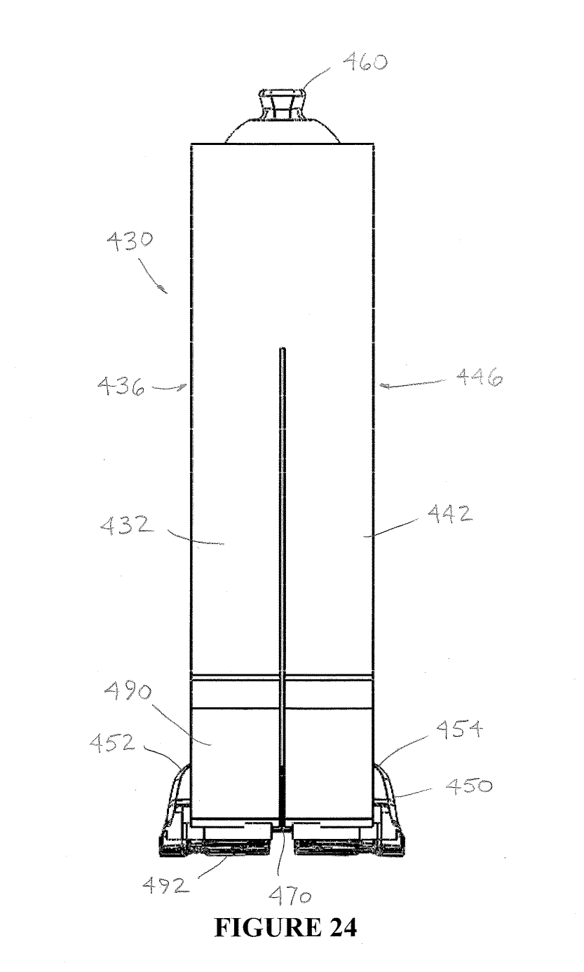

Referring now to FIG. 24, a back view of preferred split toe blade 430 is illustrated. As shown in FIG. 24, preferred split toe blade 430 comprises medial toe member 432 having rearward end 436, lateral toe member 442 having rearward end 446, toe sole 450 having medial toe sole member 452 and lateral toe sole member 454, connecting web 470, heel member 490, and heel sole 492.

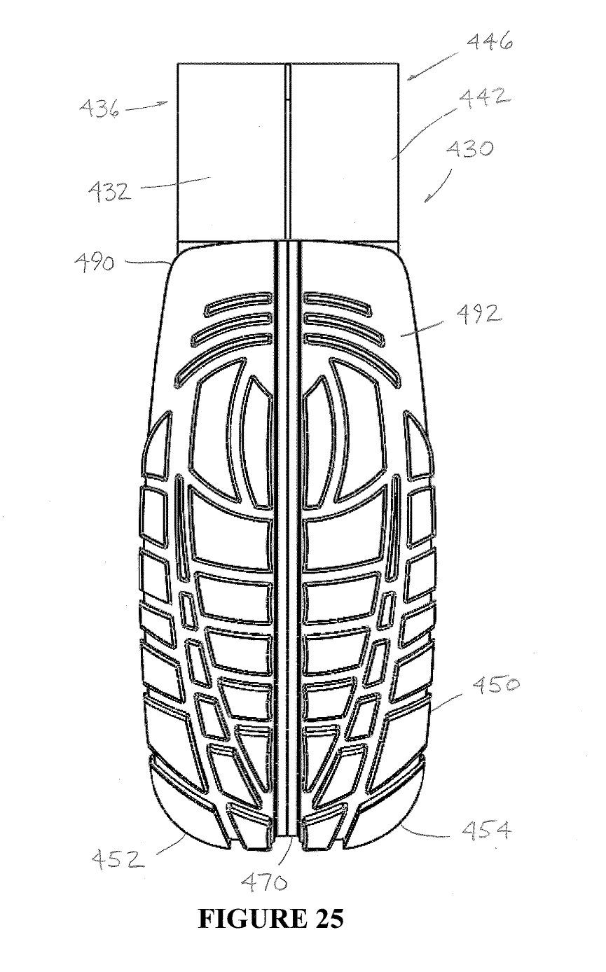

Referring now to FIG. 25, a bottom view of preferred split toe blade 430 is illustrated. As shown in FIG. 25, preferred split toe blade 430 comprises medial toe member 432 having rearward end 436, lateral toe member 442 having rearward end 446, toe sole 450 having medial toe sole member 452 and lateral toe sole member 454, connecting web 470, heel member 490, and heel sole 492. While FIGS. 22-25 illustrate the preferred configuration and arrangement of split toe blade 430, it is contemplated within the scope of the invention that the split toe blade may be of any suitable configuration and arrangement.

The invention also comprises a method for controlling the movement of a split toe blade. The preferred method comprises the step of providing a split toe blade. The preferred split toe blade comprises a medial toe member, a lateral toe member that is disposed adjacent to the medial toe member, and a connecting web that is disposed between the medial toe member and the lateral toe member. In the preferred embodiments of the split toe blade, the connecting web controls the amount of traverse compliance and prevents the medial toe member and the lateral toe member from overlapping in the event an off-axis load is applied to the blade. The preferred method also comprises the step of applying an off-axis force to the blade.

In other preferred embodiments of the method of the invention, the split toe blade also comprises a toe sole and an elastomeric and polyethylene terephthalate band, and the method also comprises the step of adjusting at least one of the connecting web and the elastomeric band.

In operation, several advantages of the preferred embodiments of the apparatus and method for a split toe blade are achieved. For example, the preferred embodiments of the apparatus and method for a split to blade are adapted to provide sufficient multi-axial performance and sufficient flexibility in the transverse plane. The preferred embodiments of the apparatus and method for a split toe blade are also adapted to provide a split toe blade that is well-suited for running on uneven terrain and other non-running activities. In addition, the preferred embodiments of the apparatus and method for a split toe blade are adapted to allow the user to turn around easily and run faster around corners. The preferred embodiments of the apparatus and method for a split toe blade are further adapted to control the amount of traverse compliance and prevent the different toe portions of the blade from overlapping in the event an off-axis load is applied.

Although this description contains many specifics, these should not be construed as limiting the scope of the invention but as merely providing illustrations of some of the presently preferred embodiments thereof, as well as the best mode contemplated by the inventors of carrying out the invention. The invention, as described herein, is susceptible to various modifications and adaptations, and the same are intended to be comprehended within the meaning and range of equivalents of the appended claims.

What is claimed is:

1. A split toe blade, said split toe blade comprising:
(a) a medial toe member;
(b) a lateral toe member, said lateral toe member being disposed adjacent to the medial toe member;
(c) a connecting web, said connecting web being disposed between the medial toe member and the lateral toe member;
wherein the connecting web controls the amount of traverse compliance in the event an off-axis load is applied to the blade.

2. The split toe blade of claim 1 wherein the connecting web comprises an elastomeric material.

3. The split toe blade of claim 1 further comprising a toe sole.

4. The split toe blade of claim 3 wherein the toe sole is disposed on at least a portion of the medial toe member and on at least a portion of the lateral toe member.

5. The split toe blade of claim 3 wherein the toe sole is disposed around at least a portion of the medial toe member and around at least a portion of the lateral toe member.

6. The split toe blade of claim 3 wherein the toe sole comprises a toe spike.

7. The split toe blade of claim 3 wherein the toe sole comprises an elastomeric material.

8. The split toe blade of claim 3 wherein the toe sole comprises medial toe sole member and a lateral toe sole member.

9. The split toe blade of claim 1 further comprising a mounting means.

10. The split toe blade of claim 9 wherein the mounting means comprises a standard modular mount.

11. The split toe blade of claim 9 wherein the mounting means comprises a posterior mount.

12. The split toe blade of claim 1 further comprising an elastomeric band.

13. The split toe blade of claim 1 further comprising a heel member.

14. The split toe blade of claim 13 further comprising a heel sole, said heel sole being disposed on at least a portion of the heel member.

15. The split toe blade of claim 14 wherein the heel sole is disposed around at least a portion of the heel member.

16. A split toe blade, said split toe blade comprising:
(a) a medial toe member;
(b) a lateral toe member, said lateral toe member being disposed adjacent to the medial toe member;
(c) a toe sole, said toe sole being disposed on at least a portion of the medial toe member and on at least a portion of the lateral toe member, and said toe sole having a connecting web disposed between the medial toe member and the lateral toe member;
wherein the connecting web controls the amount of traverse compliance in the event an off-axis load is applied to the blade.

17. A method for controlling the movement of a split toe blade, said method comprising the steps of:
(a) providing a split toe blade, said split toe blade comprising:
(i) a medial toe member;
(ii) a lateral toe member, said lateral toe member being disposed adjacent to the medial toe member;
(iii) a connecting web, said connecting web being disposed between the medial toe member and the lateral toe member;
wherein the connecting web controls the amount of traverse compliance in the event an off-axis load is applied to the blade; and
(b) applying an off-axis force to the blade.

18. The method of claim 17 wherein the split toe blade further comprises a toe sole.

19. The method of claim 17 wherein the split toe blade further comprises an elastomeric band.

20. The method of claim 17 further comprising the step of adjusting at least one of the connecting web and the polyethylene terephthalate band.
